(19) United States
(12) Patent Application Publication
(10) Pub. No.: US 2023/0270954 A1
(43) Pub. Date: Aug. 31, 2023

(54) Method for Determining Compliance of a Cavity in Minimally Invasive Surgery

(71) Applicant: W.O.M. World of Medicine GmbH, Berlin (DE)

(72) Inventors: Jan-Hendrik Hendrik Carstens, Berlin (DE); Ibrahim Ilik, Bielefeld (DE); Felix Menzel, Berlin (DE)

(73) Assignee: W.O.M. World of Medicine GmbH, Berlin (DE)

(21) Appl. No.: 18/008,225

(22) PCT Filed: Jun. 7, 2021
(86) PCT No.: PCT/DE2021/000106

§ 371 Date: Dec. 21, 2022

(30) Foreign Application Priority Data
Jun. 5, 2020 (DE) 10 2020 003 419.5

(51) Int. Cl. A61M 13/00; A61M 31/00

(52) U.S. Cl. CPC A61M 13/003; A61M 31/00; A61M 2205/3344; A61M 2205/50

(57) ABSTRACT

The invention relates to a method for determining compliance of a cavity in minimally invasive surgery and to devices for carrying out said method.

### SUBJECT MATTER OF THE INVENTION

The invention relates to a method for determining compliance of a cavity in minimally invasive surgery and to devices for carrying out said method.

### PRIOR ART

In minimally invasive surgery, there is a need for expansion of a cavity or of the surgical area in the human body. Examples of this are the abdomen or the bladder. Depending on the size of the cavity, the necessary volume of a fluid (e.g., CO₂, saline solution) varies, in order to expand this cavity such that a sufficient field of vision for the surgical invention is enabled.

In the case of gaseous fluids (such as, e.g., CO₂) devices are used that adjust, by using pressure reducers, the necessary volumetric flow, which is then supplied to the cavity.

The situation is different when using liquid fluids (such as, e.g., saline solutions). Here, e.g., peristaltic pumps are used that are able to vary the volumetric flow via the control of the pump rotation.

By introducing the volumetric flow into the cavity, the hollow space is filled with the fluid, and the pressure in this cavity increases. At the same time, the hollow space expands so far that the field of vision increases.

The so-called compliance (expansibility) of a cavity C_{c} can be determined using the relationship between volume V_{c} and resulting pressure p_{c} as a static characteristic using the equation

C_{c}=V_{c}/p_{c}

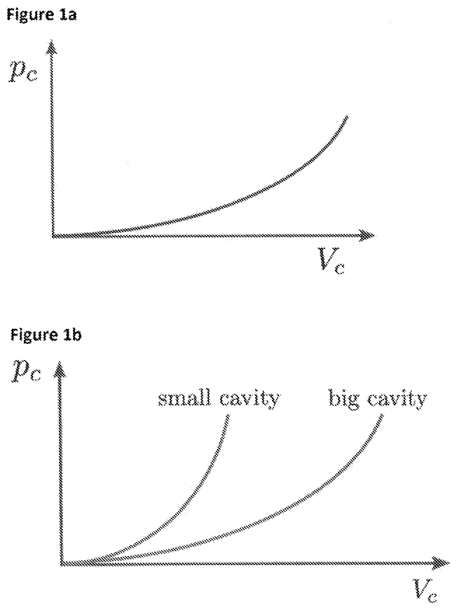

### (see FIG. 1a).

The reciprocal of the compliance C_{c} is referred to as elasticity E_{c}=1/C_{c}.

Here, the basic condition is that the specific pressure in this cavity must not have any harmful effects on the patient. For this reason, typically, pressure sensors are used for determining the cavity pressure. By a suitable regulation, the necessary volumetric flow can be calculated, without a cavity pressure harmful for the patient being caused. Accordingly, the necessary volumetric flow is realized by the regulation of the pressure reducer or the peristaltic pump. However, it must be taken into account that during the insufflation, the pressure is not measured: For the pressure measurement, the insufflation is interrupted for a short time, so that a pressure equilibrium is established, which represents the actual pressure in the body cavity. After the measurement, insufflation is continued.

Depending on the indication of the surgery and the physical characteristics of the patient (e.g., child or adult), a significant variation of the necessary volume is required, in order to inflate the body cavity to the desired pressure (see FIG. 1b).

Actually, therefore, the user of the device has to perform a multitude of necessary settings, in order to communicate the information about the intended indication and cavity size to the device.

Therefrom can be derived, in particular, the parameters and limit values for the control/regulation of the device. E.g., thus, data sets are loaded, which quantify the maximum allowable flow rate of the fluid.

When the body cavity is larger than originally assumed, then the expansion of the body cavity takes a very long time and undesired delays in the surgery will occur. When, however, the body cavity is smaller than originally assumed, then possibly very quickly pressures are achieved, which may result in tissue damage.

Another problem may arise when the attending staff misses the body cavity with the gas-carrying Veress needle. In this case, possibly, an emphysema is formed, which may be very painful.

In case the user thus sets a wrong indication and/or an assumed cavity size (e.g., by preselection of adult or child) at the device, a faulty behavior of the device may result. Herein, e.g., an unadapted limitation of the maximum flow rate could lead to an undesired time delay of the surgery or otherwise to high-pressure loads.

The prior art devices and methods are not able, up to now, to solve the described problems. The relevant prior art comprises the documents US 2007/0083126 A1, US 2010/0236555 A1, DE 4309380 A1, DE 19809867 C1, Tautorat, C. et al., Balloon-based measuring systems for compliance investigations. In: Current Directions in Biomedical Engineering 4(1), 2018.

There is therefore a need for a regulation system of a medical-technical device that automatically determines the crucial characteristics of a cavity.

### Solution According to the Invention

The present invention discloses a medical-technical device for introducing of a fluid into a body cavity, which device determines the characteristics of a cavity and thus automatically identifies the necessary operating parameters.

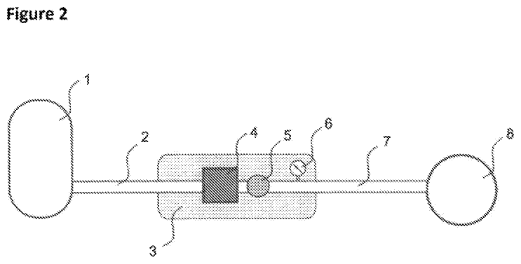

FIG. 2 shows a medical-technical device (3) according to the invention for supplying a fluid comprising the following components:

A fluid reservoir (1), from which the fluid is taken and supplied to the supply unit (4) using a connecting element (2). The fluid may be a gas (e.g., CO₂ or N₂) or a liquid (e.g., saline solution).

A regulated pump (actuator or supply unit) (4) for supplying the fluid in a regulated manner.

A measuring device for the volumetric flow (5).

A pressure sensor (6) for determining the dynamic and static pressure of the fluid.

A connecting element (7) (e.g., tube) for supplying the fluid from the device to the body cavity (8).

An electronic storage element (not explicitly shown), which serves for detecting measurement data. Further, an electronic computing unit (e.g., microcontroller) for sending necessary control commands to the actuators, evaluating data, loading/writing parameter data sets from the storage element.

By means of a medical-technical device comprising the mentioned components, the compliance of the cavity can automatically be determined using the values of volumetric flow and pressure so that operating errors of the medical staff are avoided. To this end, different methods of determination can be applied, which are described in the following.

### METHOD I.A

First, the device is connected to the body cavity through the connecting element. Then, the device is turned on. Before initially applying a volumetric flow, the device determines the pressure in the cavity. Then, a predefined temporal volumetric flow q is generated using the actuator (e.g., a pulsed volumetric flow, with a defined length in time). The volumetric flow generates a pressure increase q_{c} in the cavity.

The volume V can be determined by the integration of the volumetric flow by the measurement unit. After the defined volumetric flow, the device stops the supply and identifies the static pressure in the cavity. Thus, the elasticity can be determined using the partial pressure increase (dp_{c}/dV_{c}). This procedure can be repeated until a desired reference pressure in the cavity is achieved. From the partial pressure increases, then the so-called p-V diagram can be derived. This diagram, thus, provides information about the size of the cavity or the location of the indication. Then, by comparison to system parameters, the parameterization and selection of optimum system parameters (e.g., maximum flow rate, control, and regulation parameters) can be performed. By an optional confirmation by the user, the automatic cavity detection can be confirmed.

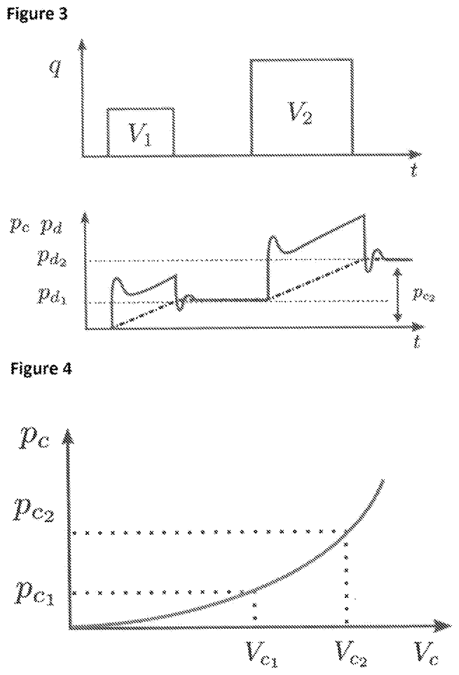

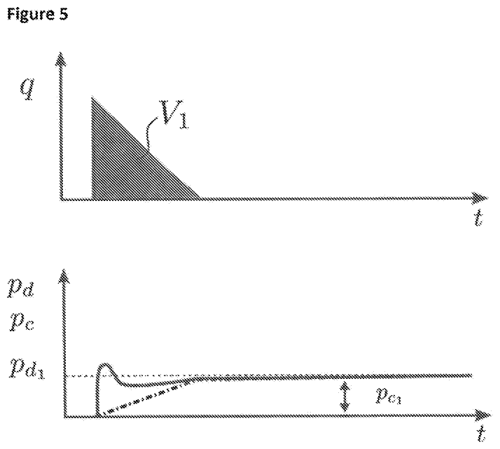

In FIG. 4, an example of this method is shown. Two volumes V₁ and V₂ are supplied temporally offset into the hollow space. Then, the pressure in the cavity p_{c} increases, and the pressure of the cavity can be determined using the pressure sensor p_{d}. This results in the working points V_{c1}=V₁, p_{c1}=p_{d1} and V_{c2}=V₂+V₁, p_{c2}=p_{d2}. By, e.g., linear approximation, then an approximation of the p-V diagram can be calculated (see FIG. 4). The “transient response” of the pressure measurement signal at the starting point and at the stopping point of the volumetric flow can clearly be seen in the measurement diagram (FIGS. 5 to 7 bottom).

### METHOD I.B

In the reality of minimally invasive interventions, often leakages in the cavities occur. Such leakages falsify the procedure in Method I.a due to this fluid outflow of an unknown quantity. In order to compensate for the influence of the leakage in the measurement data, Method I.a is extended as follows:

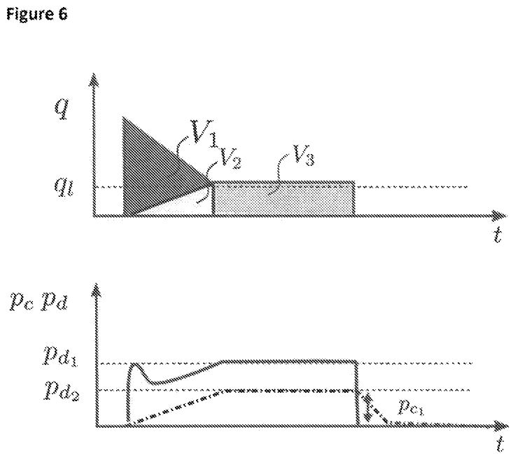

By a pressure regulation device, a pressure is generated in the cavity. In this case, the volumetric flow necessary for achieving the desired pressure is predefined. In a closed cavity—without leakage—, the pressure regulation device would regulate the volumetric flow to zero when the desired pressure is achieved (see FIG. 6).

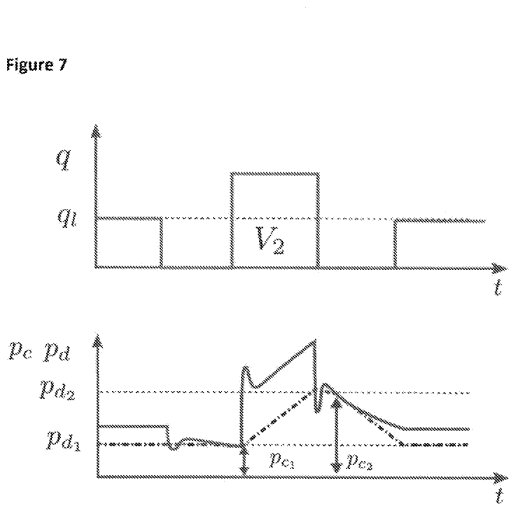

With an existing leakage in the body cavity, the pressure regulation system would permanently adjust a volumetric flow, in order to compensate for the leakage. This volumetric flow, which is necessary to maintain the pressure, is the leakage volumetric flow qₗ for the present cavity pressure. This is exemplarily shown in FIG. 7. Therefrom, the volumes V₂ and V₃, which leave the body cavity through the leakage, can be determined. Then, the introduced volume can be cleared from the leakage.

The pressure in the cavity p_{c1} at the time when the volumetric flow is stopped can be determined or approximated through prior knowledge of the pressure drop across the connecting element and the measured pressure p_{d1}. At this time is p_{d}≈p_{c1}.

Herein, the evaluation can be applied as in Method I.a. In order to allow for several working points for the calculation of the p-V diagram, the reference pressure can be increased (temporarily).

By repetition for other reference pressure values, different working points of the p-V diagram and thus the cavity size can be determined.

### METHOD II

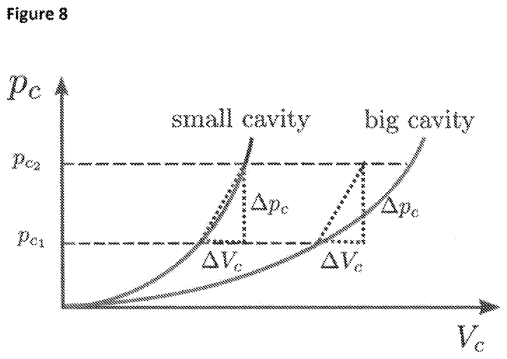

During the operation of the device, the actual working point in the p-V diagram of the body cavity can be determined. A low partial capacity value (ΔC_{c}=ΔV_{c}/Δp_{c}) suggests a large body cavity, or a large value suggests a small body cavity. In order to obtain this information, a measurement pause is generated during the operation of the device. Herein, the volumetric flow rate is briefly interrupted, and the stationary cavity pressure p_{c1} is identified. Then, a predefined temporal volumetric flow is generated using the actuator (e.g., a pulsed volumetric flow with a defined length in time). The volumetric flow generates a pressure increase in the cavity. The volume V₂ supplied in this period can be determined by the integration of the volumetric flow by the measurement unit. After the defined volumetric flow, the device stops the supply and identifies the static pressure in the cavity p_{c2}. Then, the device resumes its normal functionality (see FIG. 7). From the measurements results ΔC_{c}=V₂/(p_{c2}−p_{c1}). Different from Method I is that no complete information about the cavity size or the p-V diagram is known. Thus, this information only applies to the actual working point of the volumetric flow, which is necessary for maintaining the cavity pressure. However, in this working point, the plausibility for the selected default setting by the user and actually determined characteristic values can be adjusted (see FIG. 8). In the case of a discrepancy, thus, the device can automatically adjust the parameter set of the device, in order to allow the user an optimum system setup for carrying out the intervention.

### METHOD III

During the operation of the device, the pressure is temporarily increased. To this end, an active pressure control/regulation is used. The necessary additional volume for obtaining the desired pressure in the cavity is determined in the phase of the pressure increase. Therefrom, the partial capacity value (ΔC=ΔV/Δp) can be determined. This is identical to the procedure in Method II. However, Method III can also be used in the initial filling phase of the cavity. To this end, the desired reference pressure of the pressure regulation is increased quasi-stationarily (very slowly in time or step-by-step). A measurement pause is not necessary with the present system parameters for the device and the connecting unit between the device and the body cavity. The data of the volume and the generated pressure can thus be transferred into a p-V diagram. This provides, same as in Method I, the basis for deriving the cavity size or indication. Thus, the possibility to perform a parameterization and selection of optimum system parameters (e.g., maximum flow rate, control, and regulation parameters) will result. By an optional confirmation by the user, the automatic cavity detection can be confirmed.

### METHOD IV

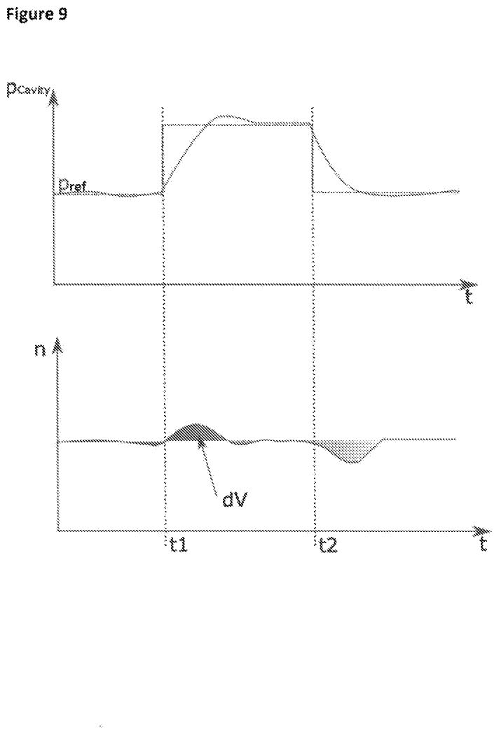

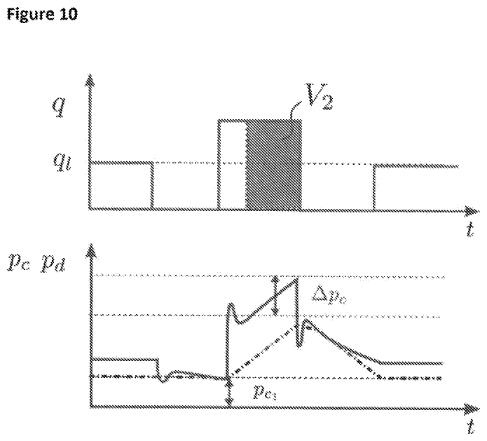

In a variation of Method II, the volumetric flow is increased after the determination of the actual cavity pressure p_{c1}. The rising pressure at the sensor correlates with the pressure rise in the cavity (see FIG. 9). Therefrom results that a measurement of the cavity pressure p_{c2} is not necessary (comp. Method II). Instead (see FIG. 10), the increase Δp_{c} relative to the volume V₂ is identified. After the determination of the values, the device resumes the previous operation.

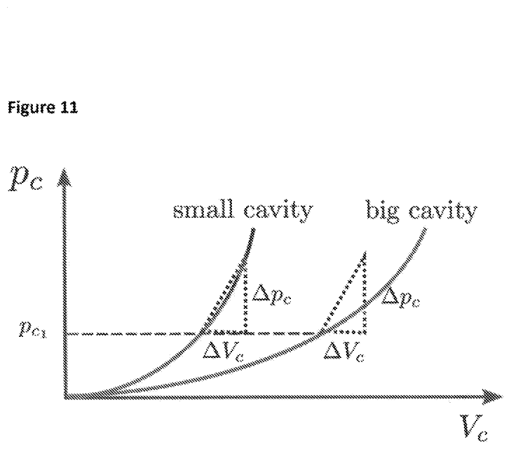

In contrast to Method II, thus, it lacks the exact knowledge of the value of the cavity pressure p_{c2}, however, the same partial increases will result, and thus, the value can be used by the user for comparison of the parameter set of the device to the determined cavity values (FIG. 11) and be modified if necessary, in order to guarantee an optimum parameterization of the device.

### LIST OF REFERENCES

(1) fluid reservoir
(2) fluid connection (supply tube of the fluid between reservoir and medical-technical device for supplying a fluid (3)
(3) medical-technical device for supplying fluids
(4) supply device
(S) measuring device for the volumetric flow of the fluid
(6) pressure sensor
(7) fluid connection
(8) body cavity

1. A method for determining compliance of a cavity C_{c} using a medical-technical device by
a) controlled introduction of a fluid,
b) single or multiple measurements of the volume introduced into the cavity and of the cavity pressure resulting therefrom,
c) calculation of the compliance C_{c} using the equation C_{c}=V_{c}/p_{c}.

2. The method for determining compliance of a cavity according to claim 1, characterized by a temporally offset introduction of at least two defined fluid volumes into the cavity and consequent calculation of the partial pressure increase (dp_{c}/dV_{c}).

3. The method for determining compliance of a cavity according to claim 1, characterized by the determination of the leakage volumetric flow qₗ before the single or multiple measurements of the volume introduced into the cavity and of the cavity pressure resulting therefrom and by taking into account the leakage volumetric flow qₗ when calculating C_{c} using the equation (ΔC_{c}=(ΔV_{c}−qₗ)/Δp_{c})I.

4. A medical-technical device for determining compliance of a cavity C_{c}, comprising the components
at least one fluid reservoir (1), from which the fluid is taken and supplied to the supply unit (4) through the connecting element (2),
at least one regulated pump (actuator or supply unit) (4) for supplying the fluid in a regulated manner,
at least one measuring device (5) for the volumetric flow of the fluid,
at least one pressure sensor (6) for determining the dynamic and static pressure of the fluid,
at least one connecting element (7) (e.g., tube) for supplying the fluid from the device to the body cavity (8),
at least one electronic storage element, which serves for detecting measurement data,
at least one electronic computing unit (e.g., microcontroller}, for supplying necessary control commands to the actuators, to carry out the determination method according to claim 1 and to load parameter data sets from the storage element or to write them on the storage element.
